(19) United States
(12) Patent Application Publication
(10) Pub. No.: US 2024/0382478 A1
(43) Pub. Date: Nov. 21, 2024

(54) PIMOBENDAN FORMULATION AND METHOD OF USE THEREOF

(71) Applicant: Dechra Veterinary Products, LLC, Overland Park, KS (US)

(72) Inventors: Douglas I. Hepler, Overland Park, KS (US); Michael S. Daniel, Overland Park, KS (US); Neil E. Paulsen, Overland Park, KS (US); Gail L. Dempsey, Overland Park, KS (US)

(21) Appl. No.: 18/786,386

(22) Filed: Jul. 26, 2024

Related U.S. Application Data
(63) Continuation of application No. 17/079,258, filed on Oct. 23, 2020, now abandoned.
(60) Provisional application No. 62/924,985, filed on Oct. 23, 2019.

(51) Int. Cl. A61K 31/501; A61K 9/16; A61K 9/50

(52) U.S. Cl. CPC A61K 31/501; A61K 9/1611; A61K 9/1617; A61K 9/1623; A61K 9/1652; A61K 9/1664; A61K 9/5026; A61K 9/5031

(57) ABSTRACT

Provided herein are chewable oral formulations including pimobendan for use in mammals to treat cardiac disorders.

### CROSS-REFERENCE TO RELATED APPLICATIONS

This application is a continuation application of U.S. application Ser. No. 17/079,258 filed Oct. 23, 2020, now pending; which claim the benefit under 35 USC § 119 (e) to U.S. Application Ser. No. 62/924,985 filed Oct. 23, 2019, now expired. The disclosure of each of the prior applications is considered part of and is incorporated by reference in the disclosure of this application.

### BACKGROUND OF THE INVENTION

### Field of the Invention

The invention relates generally to pharmaceutical formulations and more specifically to oral formulations including pimobendan for use in mammals to treat cardiac disorders.

### BACKGROUND INFORMATION

Pimobendan, (4,5-dihydro-6-[2-(4-methoxyphenyl)-1H-benzimidazol-5-yl]-5-methyl-3(2H)-pyridazinone) is disclosed in U.S. Pat. No. 4,361,563, herein incorporated by reference in its entirety.

Pimobendan is a cardiotonic, hypotensive and anti-thrombotic therapeutic and is useful for the treatment of cardiac disorders, such as congestive heart failure in mammals. Pimobendan is an inodilator compound with calcium sensitizing effects, as well as some phosphodiesterase type III inhibitory effects. Rather than increasing calcium entry into cardiac myocytes, calcium sensitizers achieve their positive inotropic effect by sensitizing the contractile proteins to existing cytosolic calcium, by altering the binding of calcium with troponin-C. Producing a positive inotropic effect by calcium sensitizing thereby avoids some of the adverse effects of cytosolic calcium overload. Increased cytosolic calcium levels have been associated with an increased tendency for arrhythmias and sudden death. Clinical trials of long-term use of oral pimobendan in human patients with heart failure have demonstrated an improvement in exercise tolerance and quality of life without significant adverse effects on survival.

Cardiac disorders are a problem known to occur in small mammals, such as cats and dogs, as well as humans. For example, hypertrophic cardiomyopathy is the most common heart disease in cats and the most common cause of heart failure in this species. Additionally, intravenous positive inotropic agents play a vital role in the management of acute heart failure and will often result in a short-term improvement in dogs with dilated cardiomyopathy.

It is desirable to have formulations which provide prolonged therapeutic relief to a mammal for cardiac disorders which also exhibit extended shelf stability.

### SUMMARY OF THE INVENTION

In one aspect, the disclosure provides a single dose, chewable oral formulation including pimobendan or a pharmaceutically acceptable salt or solvate thereof; and a pharmaceutically acceptable carrier. The pimobendan is in a granular form and coated with a polyvinyl alcohol-polyethylene glycol graft copolymer. Additionally, the formulation is stable for at least 6, 12, 18, 24, 30, or 36 months or greater at 25° C. or 40° C.

In embodiments, the pimobendan granules of the formulation are formed by mixing pimobendan with lactose and/or dicalcium phosphate, granulating the mixture to produce granules with a dimension having an average size of between about 100 and 1500 μm, about 100 and 1000 μm, or about 200 and 850 μm, and then coating the granules with a polyvinyl alcohol-polyethylene glycol graft copolymer. In embodiments the granules have a dimension with an average size of less than about 1200, 1100, 1000, 900, 800, 700, 600, 500, 400, 300 or 200 μm.

In embodiments, the formulation is a homogenous mixture including coated pimobendan granules dispersed in a pharmaceutically acceptable carrier which includes starch, croscarmellose sodium, sodium lauryl sulfate, magnesium stearate, dicalcium phosphate, polyethylene glycol, glycerin, soybean oil and optionally one or more flavorings, antioxidants and/or polyvinylpyrrolidone.

Also provided herein is a method of treating a cardiac disease or disorder in a subject by administering a therapeutically effective amount of a formulation of the disclosure. In embodiments the subject is a mammal, such as a canine or feline. In various embodiments cardiac disease or disorder is congestive heart failure, cardiomyopathy, dilated or restrictive cardiomyopathy or atrioventricular valvular insufficiency.

### BRIEF DESCRIPTION OF THE DRAWINGS

FIG. 1 is a graphical representation depicting data in one embodiment of the disclosure.

FIG. 2 is a graphical representation depicting data in one embodiment of the disclosure.

### DETAILED DESCRIPTION OF THE INVENTION

The following terms, definitions and abbreviations apply. Abbreviations used herein have their conventional meaning within the chemical and biological arts.

The term “subject” refers to mammalian organisms to be treated by the methods of the disclosure. Such organisms include, but are not limited to, companion animals such as domestic dogs and cats. In the context of the disclosure, the term “subject” generally refers to an individual who will receive or who has received treatment described below (e.g., administration of the compositions of the disclosure).

As used herein, a “patient” or “subject” refers to either a human or non-human mammalian animal. Non-human animals include any non-human mammalian animals. Such non-human animals may include, but are not limited to rodents, non-human primates (e.g., monkey and apes), ungulates, ovines, bovines, ruminants, lagomorphs, porcines, caprines, equines, canines, felines, murines, and the like. In certain embodiments of the disclosure, the animals are mammals. In some embodiments, the animals include, but are not limited to, companion animals such as domestic dogs and cats. In the context of the disclosure, the term “subject” generally refers to an individual who will receive or who has received treatment described below (e.g., administration of a composition of the disclosure).

The term “therapeutically effective amount” means the amount of the compound or pharmaceutical composition that will elicit the biological or medical response of a patient or tissue that is being sought by the researcher, veterinarian, medical doctor or other clinician.

By “pharmaceutically acceptable” it is meant the carrier, diluent or excipient must be compatible with the other ingredients of the formulation and not deleterious to the recipient thereof.

The terms “administration of” and/or “administering a” compound should be understood to mean providing a compound of the disclosure or pharmaceutical composition to the subject in need of treatment.

The term “about” with respect to a number means that the number includes a range of insignificant variation above and below the number unless otherwise stated; e.g., a value of 1 will be understood to include up to 0.5 to 1.5 and all numbers thereinbetween.

In embodiments, the pharmaceutical formulations of the disclosure are in the form of an oral dosage, such as a tablet or capsule, including pimobendan and a pharmaceutical carrier.

To provide enhanced stability of the formulation, pimobendan is incorporated into the formulation in a granular form and coated a polyvinyl alcohol-polyethylene glycol graft copolymer, such as Kollicoat® IR. As such, the formulation of the disclosure is stable for at least 6, 12, 18, 24, 30, or 36 months or greater at 25° C. or 40° C.

In embodiments, the pimobendan granules of the formulation are formed by mixing pimobendan with lactose and/or dicalcium phosphate, granulating the mixture to produce granules with a dimension having an average size of between about 100 and 1500 μm, about 100 and 1000 μm, or about 200 and 850 μm, and then coating the granules with a polyvinyl alcohol-polyethylene glycol graft copolymer. In embodiments the granules have a dimension with an average size of less than about 1200, 1100, 1000, 900, 800, 700, 600, 500, 400, 300 or 200 μm.

In embodiments, the pimobendan granules are formed by mixing pimobendan with lactose and/or dicalcium phosphate and granulating the mixture by passage through a #20mesh screen having openings of 850 μm. The resulting granules are then coated with a polyvinyl alcohol-polyethylene glycol graft copolymer.

In embodiments, a solvent is utilized during production of the pimobendan granules but which is not present in the resulting granule that is coated. For example, a solvent, such as water or ethanol, may be used to dissolve a suitable binder, such as polyvinylpyrrolidone, before being added to a dry mixture of pimobendan and lactose monohydrate and/or dicalcium phosphate. While mixing the dry mixture of pimobendan and lactose and/or dicalcium phosphate, the binder solution is added. Once the binder solution is added and the resulting mixture is sufficiently mixed, it is passed through a suitable mesh screen (a #20 mesh) which produces appropriately sized granules. At this point the granules are still wet and allowed to dry by evaporating off the solvent before coating.

Once the solvent is evaporated off and the granules are dried, they are coated with a polyvinyl alcohol-polyethylene glycol graft copolymer. The coating is then allowed to dry and the dried coated granules are compounded with a pharmaceutically acceptable carrier.

In certain embodiments, the formulation of a pimobendan granule is as set forth in Table I below.

TABLE I
Pimobendan Coated Granule Formulation

| Component | w/w % |
|---|---|
| Pimobendan | 1-15 |
| Dicalcium Phosphate | 70-90 |
| Polyvinylpyrrolidone | 1-10 |
| polyvinyl alcohol-polyethylene glycol graft | 0-10 |
| copolymer coating (Kollicoat IR ®) | |
| | 100 |

In certain embodiments, the formulation of a pimobendan granule is as set forth in Table II below.

TABLE II
Pimobendan Coated Granule Formulation

| Component | w/w % |
|---|---|
| Pimobendan | 4-5 |
| Dicalcium Phosphate | 85-90 |
| Polyvinylpyrrolidone | 1-5 |
| polyvinyl alcohol-polyethylene glycol | 3-7 |
| graft copolymer coating (Kollicoat IR ®) | |
| | 100 |

In certain embodiments, the formulation of a pimobendan granule is as set forth in Table III below.

TABLE III
Pimobendan Coated Granule Formulation

| Component | w/w % |
|---|---|
| Pimobendan | 4-5 |
| Dicalcium Phosphate | 87 |
| Polyvinylpyrrolidone | 3 |
| polyvinyl alcohol-polyethylene glycol graft | 5 |
| copolymer coating (Kollicoat IR ®) | |
| | 100 |

In certain embodiments, the formulation of a pimobendan granule is as set forth in Table IV below.

TABLE IV
Pimobendan Coated Granule Formulation

| Component | w/w % |
|---|---|
| Pimobendan | 1-15 |
| Lactose monohydrate | 70-90 |
| Polyvinylpyrrolidone | 1-10 |
| polyvinyl alcohol-polyethylene glycol graft | 0-10 |
| copolymer coating (Kollicoat IR ®) | |
| | 100 |

In certain embodiments, the formulation of a pimobendan granule is as set forth in Table V below.

TABLE V
Pimobendan Coated Granule Formulation

| Component | w/w % |
|---|---|
| Pimobendan | 4-5 |
| Lactose monohydrate | 85-90 |
| Polyvinylpyrrolidone | 1-5 |
| polyvinyl alcohol-polyethylene glycol graft | 3-7 |
| copolymer coating (Kollicoat IR ®) | |
| | 100 |

In certain embodiments, the formulation of a pimobendan granule is as set forth in Table VI below.

TABLE VI
Pimobendan Coated Granule Formulation

| Component | w/w % |
|---|---|
| Pimobendan | 4-5 |
| Lactose monohydrate | 87 |
| Polyvinylpyrrolidone | 3 |
| polyvinyl alcohol-polyethylene glycol graft | 5 |
| copolymer coating (Kollicoat IR ®) | |
| | 100 |

In one embodiment of the disclosure, the oral compositions are in the form of a soft chewable formulation (“soft chew”) which is palatable and acceptable to the animal. The formulation is a homogenous mixture including pimobendan coated granules dispersed in a pharmaceutically acceptable carrier.

Pimobendan, (4,5-dihydro-6-[2-(4-methoxyphenyl)-1H-benzimidazol-5-yl]-5-methyl-3(2H)-pyridazinone), for use in the formulation of the disclosure is described in U.S. Pat. No. 4,361,563, herein incorporated by reference in its entirety. It will be appreciated that pimobendan may be formulated into the formulation of the disclosure in natural or salt forms. Pharmaceutically acceptable non-toxic salts include the base addition salts (formed with free carboxyl or other anionic groups), which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure include amine salts formed by the protonation of an amino group with inorganic acids such as hydrochloric acid, hydrobromic acid, hydroiodic acid, sulfuric acid, phosphoric acid, and the like. Salts of the disclosure may also include amine salts formed by the protonation of an amino group with suitable organic acids, such as p-toluenesulfonic acid, acetic acid, and the like.

In addition to the pimobendan, the formulations of the disclosure may include one or more of the following components: a solvent or mixture of solvents, one or more fillers, one or more binders, one or more surfactants, one or more humectants, one or more lubricants, one or more disintegrants, one or more colorants, one or more antimicrobial agents, one or more antioxidants, one or more pH modifiers and one or more flavoring agents.

Preferably, the components of the oral compositions will be classified as food grade quality or higher (e.g., USP or NF grade). The term “food grade” is used to refer to material that is suitable for consumption by animals and will not contain chemical or other agents that are hazardous to the health of the animal. Thus, a food grade component, if of animal origin, will be prepared to substantially reduce or eliminate the presence of infectious agents or contaminants by processes known in the art such as pasteurization, filtration, pressurization or irradiation. More preferably, the components of the oral compositions of the disclosure will not be of animal origin to avoid transmission of infective agents.

Various fillers known in the art may be used in the soft chewable compositions of the disclosure. Fillers include, but are not limited to, dicalcium phosphate, corn starch, pre-gelatinized corn starch, soy protein fines, corn cob, and corn gluten meal, and the like.

In embodiments, the filler is native starch, gelatinized starch, partly gelatinized starch, starch powder, starch granules, chemically modified starch, swellable physically modified starch or mixtures thereof. In some embodiments, a combination of two or more fillers may be used in the compositions.

The starch component may comprise starch from any source and may act as a binder in the soft chew. In one embodiment, the starch component used in the compositions is unmodified. In another embodiment, the starch component is derivatized and/or pregelatinized. In another embodiment, the starch component is highly derivatized. Some starches that can serve as a base starch for derivatization include regular corn, waxy corn, potato, tapioca, rice, etc. Suitable types of derivatizing agents for the starch include, but are not limited to, ethylene oxide, propylene oxide, acetic anhydride, and succinic anhydride, and other food approved esters or ethers, introducing such chemicals alone or in combination with one another.

In various embodiments, prior cross-linking of the starch in the starch component may or may not be necessary, based on the pH of the system and the temperature used to form the product.

The starch component may also include amylaceous ingredients. The amylaceous ingredients can be gelatinized or cooked before or during the forming step to achieve the desired matrix characteristics. If gelatinized starch is used, it may be possible to prepare the product of the subject disclosure or perform the process of the subject disclosure without heating or cooking. However, ungelatinized (ungelled) or uncooked starch may also be used.

Fillers are typically present in the compositions at a concentration of about 5% to about 80% (w/w), about 10% to about 70% (w/w), about 10% to about 60%, about 10% to about 50% (w/w), or about 10% to about 40% (w/w). More typically, the fillers may be present at concentrations of about 10% to about 40% (w/w), about 10% to about 30% (w/w), about 10% to about 25% (w/w) or about 15% to about 25% (w/w).

Binders that may be used in the compositions of the disclosure include, but are not limited to, polyvinylpyrrolidone (e.g., Povidone), cross-linked polyvinylpyrrolidone (Crospovidone), polyethylene glycols of various grades including PEG 3350, PEG 4000,PEG 6000, PEG 8000 and even PEG 20,000, and the like; co-polymers of vinylpyrrolidone and vinyl acetate (e.g., Copovidone) such as the product sold by BASF by the tradename Kollidon® VA 64 and the like; starch such as potato starch, tapioca starch or corn starch; molasses, corn syrup, honey, maple syrup and sugars of various types; or a combination of two or more binders. In one embodiment, the composition comprises the binders Povidone K30 LP and PEG 3350 or PEG 4000, or a combination thereof. Binders are typically present in the compositions at a concentration of about 1% to about 30% (w/w). More typically, the compositions will include binders at a concentration of about 1% to about 20% (w/w), about 1 to about 15% (w/w), about 1% to about 10% (w/w), about 1% to about 5% (w/w) or about 1% to about 3% (w/w).

Humectants that may be used in the compositions include, but are not limited to, glycerol (also referred to herein as glycerin), propylene glycol, cetyl alcohol and glycerol monostearate, and the like. Polyethylene glycols of various grades may also be used as humectants.

In some embodiments, the humectant may comprise more than one oil including, but not limited to, fat or fats, both natural and synthetic. Oil employed as an ingredient in the soft chew may be a saturated or unsaturated liquid fatty acid, its glyceride derivatives or fatty acid derivatives of plant or animal origin or a mixture thereof. A source for typical animal fats or oils are fish oil, chicken fat, tallow, choice white grease, prime steam lard and mixtures thereof. However, other animal fats are also suitable for use in the soft chew. Suitable sources for vegetable fats or oils can be derived palm oil, palm hydrogenated oil, corn germ hydrogenated oil, castor hydrogenated oil, cotton-seed oil, soybean oil, olive oil, peanut oil, palm olein oil, Cacao fat, margarine, butter, shortening and palm stearin oil, and mixtures thereof. Additionally, a mixture of animal or vegetable oils or fats is suitable for use in the matrix.

Humectants may typically present in the compositions at a concentration of about 1% to about 45% (w/w). Typically, the concentration of the humectant in the composition of the disclosure will be 5% to about 40% (w/w), about 5% to about 35% (w/w) or about 10% to about 35% (w/w). More typically, the compositions of the disclosure will contain about 25% to about 35% (w/w) humectant.

Surfactants may be present in the composition at concentrations of about 0.1% to about 10% (w/w), about 1% to about 5% (w/w) or about 1% to about 3% (w/w). More typically, surfactants may be present at concentrations of about 0.05% to about 2% (w/w) or about 0.05 to about 1% (w/w). Examples of surfactants that may be used in the compositions include, but are not limited to, glyceryl monooleate, polyoxyethylene sorbitan fatty acid esters, sorbitan esters including sorbitan monooleate (Span® 20), polyvinyl alcohol, polysorbates including polysorbate 20 and polysorbate 80, d-.alpha.-tocopheryl polyethylene glycol 1000 succinate (TPGS), sodium lauryl sulfate, co-polymers of ethylene oxide and propylene oxide (e.g., poloxomers such as LUTROL® F87 and the like), polyethylene glycol castor oil derivatives including polyoxyl 35 castor oil (Cremophor® EL), polyoxyl 40 hydrogenated castor oil (Cremophor® RH 40), polyoxyl hydrogenated castor oil (Cremophor® RH60); propylene glycol monolaurate (LAUROGLYCOL®); glyceride esters including glycerol caprylate/caprate (CAPMUL® MCM), polyglycolized glycerides (GELUCIRER), PEG 300 caprylic/capric glycerides (Softigen® 767), PEG 400caprylic/capric glycerides (Labrasol®), PEG 300 oleic glycerides (Labrafil® M-1944CS), PEG 300 linoleic glycerides (Labrafil® M-2125CS); polyethylene glycol stearates and polyethylene glycol hydroxy stearates including polyoxyl 8 stearate (PEG 400 monostearate), polyoxyl 40 stearate (PEG 1750 monostearate, and the like. Polyethylene glycol stearates (synonyms include macrogol stearates, polyoxylstearates, polyoxyethylene stearates, ethoxylated stearates; CAS No. 9004-99-3, 9005-08-7) are mixtures of mono- and distearate esters of mixed polyoxyethylene polymers. Polyethylene glycol hydroxystearate is a mixture of mono- and diesters of hydroxystearic acid with polyethylene glycols. One polyethylene glycol hydroxystearate that may be used in the compositions is polyethylene glycol 12-hydroxystearate. In another embodiment, the compositions may include the surfactant polyethylene glycol 15 12-hydroxystearate (Solutol® HS 15 from BASF), a mixture of mono- and diesters of 12-hydroxystearic acid with 15 moles of ethylene oxide. Again, these compounds, as well as their amounts are well known in the art. In another embodiment of the disclosure, the compositions may include polyoxyl 35 castor oil (Cremophor® EL) as a surfactant. In other embodiments, the chewable compositions may include polyoxyl 40hydrogenated castor oil (Cremophor® RH 40) or polyoxyl 60 hydrogenated castor oil (Cremophor® RH60) as surfactants. The compositions of the disclosure may also include a combination of surfactants.

In some embodiments, the compositions of the disclosure may contain one or more disintegrants. Examples of disintegrants that may be used in the compositions of the disclosure include, but are not limited to, cellulose, carboxymethyl cellulose calcium, carboxymethyl cellulose sodium, polacrilin potassium, starch, hydroxypropyl starch, corn starch, pregelatinized starch, modified starch, lactose monohydrate, croscarmellose sodium, hydroxypropyl cellulose, glycine, crospovidone, magnesium aluminum silicate, sodium starch glycolate, guar gum, colloidal silicon dioxide, polyvinylpyrrolidone (Povidone), alginic acid, sodium alginate, calcium alginate, methylcellulose, chitosan, and the like, or a combination thereof.

In certain embodiments, the oral compositions of the disclosure will include up to about 15% (w/w) of one or more disintegrants. In one embodiment, the compositions may include about 1% (w/w) to about 12% (w/w) of one or more disintegrants. In another embodiment, the compositions may include about 1% (w/w) to about 10% (w/w) or about 5% (w/w) to about 10% (w/w) of one or more disintegrants.

The formulations may contain other inert ingredients such as antioxidants, preservatives, or pH stabilizers. These compounds are well known in the formulation art. Antioxidants may be added to the compositions of the disclosure to inhibit degradation of the active agents. Suitable antioxidants include, but are not limited to, alpha tocopherol, ascorbic acid, ascrobyl palmitate, fumaric acid, malic acid, sodium ascorbate, sodium metabisulfate, n-propyl gallate, BHA (butylated hydroxy anisole), BHT (butylated hydroxy toluene) monothioglycerol, propyl gallate, MTG (monothioglycerol), tri-ethyl citrate, citric acid, TBHQ (tert-butyl hydroquinone) and the like. The antioxidants are generally added to the formulation in amounts of from about 0.0001 to about 2.0% (w/w), based upon total weight of the formulation, for example about 0.0002 to about 1.0% or about 0.0002% to about 0.03% (w/w).

The compositions may also include anti-microbial agents or preservatives. Suitable preservatives include, but are not limited to, the parabens (methylparaben and/or propylparaben), benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. The concentration of the preservatives in the compositions of the disclosure are typically from about 0.01 to about 5.0% (w/w), about 0.01 to about 2% (w/w) or about 0.05 to about 1.0% (w/w). In one embodiment, the compositions of the disclosure will contain about 0.1% to about 0.5% (w/w) of the preservative.

Many flavoring agents may be used in the compositions of the disclosure to improve the palatability of the oral formulations. Preferred flavoring agents are those that are not derived from animal sources. In various embodiments, flavoring components derived from fruit, meat (including, but not limited to pork, beef, chicken, fish, poultry, and the like), vegetable, cheese, liver, cheeseburger, liver cheeseburger, bacon, cheese-bacon and/or artificial flavorings may be used. A flavoring component is typically chosen based upon consideration related to the organism that will be ingesting the soft chew. For example, a horse may prefer an apple flavoring component, while a dog may prefer a meat flavoring component. Although flavoring components derived from non-animal sources are preferred, in some embodiments, natural flavors containing beef or liver extracts, etc., may be used such as braised beef flavor artificial powdered beef flavor, roast beef flavor and corned beef flavor among others.

Non-animal flavoring agents include, but are not limited to, artificial beef flavors, flavors derived from plant proteins such as soy protein to which artificial flavoring has been added (e.g., soy-derived bacon flavoring), and flavors derived from plant proteins such as soy protein with no artificial flavoring.

In another embodiment, the flavoring component include, but is not limited to, strawberry flavor, tutti fruity flavor, orange flavor, banana flavor, mint flavor, and an apple-molasses.

A particularly preferred flavoring for use in the disclosure is Provesta® 356, made by Ohly, Inc. It is a light tan, water-soluble powder that builds on the properties of yeast extracts and reaction flavors to provide a pleasant smoky, cured bacon flavor. Provesta® 356contains no animal derived ingredients.

The compositions of the disclosure may include one or more flavoring agents in an amount that provides the desired level of palatability to the target animal. The one or more flavoring agents will typically be present in a concentration of about 5% to about 40% (w/w). More typically, the flavoring agents will be present in a concentration of about 10% to about 30%, or about 15% to about 25% (w/w).

In various embodiments, the oral compositions of the disclosure may be coated. Any suitable coating may be used. In an embodiment, a coating is chosen that will not interfere with an additive. In another embodiment, an additive is chosen that can modify the time for digestion of the additive(s), thereby at least partially controlling the release of the additive(s). Suitable coatings include, but are not limited to, and may be any pharmaceutically acceptable, and/or neutraceutically acceptable coating, as is common in the art (polymers, monomers). Reference can be had to U.S. Pat. No. 6,498,153, incorporated herein by reference, for a list of polymers that can function as coatings.

In other embodiments, coatings for the oral formulations include gelatin, glyceryl behenate, cocoa butter, and beeswax. Other coatings would be known to a practitioner in this art. Coatings for tablets include sugar coatings, such as seal coatings, subcoatings, and syrup coatings, as well as film coatings, such as pan-pour coatings and pan spray coatings. As well known to a practitioner of this art, the coatings contain additional components such as solvents, plasticizers, colorants, opaquant-extenders and film formers.

In embodiments, the pharmaceutically acceptable carrier includes starch, croscarmellose sodium, sodium lauryl sulfate, magnesium stearate, dicalcium phosphate, polyethylene glycol, glycerin, soybean oil and optionally one or more flavorings, antioxidants and/or polyvinylpyrrolidone.

An exemplary formulation is as set forth in Table VII below.

TABLE VII
Formulation

| Component | w/w % |
|---|---|
| Pimobendan coated granule | 5-10 |
| of Table III or VI | |
| Starch (fully pregelatinized) | 15-25 |
| Crosscarmellose sodium | 3-7 |
| Polyvinylpyrrolidone | 2-6 |
| Provesta ® flavoring | 10-25 |
| Magnesium stearate | 1-2 |
| Sodium lauryl sulfate | 0.05-0.15 |
| Glycerin | 18-27 |
| Soybean oil with 0.02% TBHQ | 7-12 |
| Polyethylene glycol 3350 | 1-3 |
| Dicalcium phosphate | 10-15 |
| | 100 |

An exemplary formulation is as set forth in Table VIII below.

TABLE VIII
Formulation

| Component | w/w % |
|---|---|
| Pimobendan coated granule of Table III or VI | 8-9 |
| Starch (fully pregelatinized) | 20 |
| Crosscarmellose sodium | 5 |
| Polyvinylpyrrolidone | 4 |
| Provesta ® flavoring | 15 |
| Magnesium stearate | 1.5 |
| Sodium lauryl sulfate | 0.1 |
| Glycerin | 22 |
| Soybean oil with 0.02% TBHQ | 9 |
| Polyethylene glycol 3350 | 2 |
| Dicalcium phosphate | 13-14 |
| | 100 |

Exemplary formulations are as set forth in Table IX below.

TABLE IX
Formulations

| Pimobendan (5mg) SCTs | | Comments |
|---|---|---|
| Identifier | Formula (% w/w) | Comments |
| PAH-19- | 4.33% Pimobendan Granule of any of Tables I-VI | Pimo 5 mg SCT |
| 01-001 | 16.00% pre-gelatinized Starch | |
| | 5.00% Croscarmellose Sodium | |
| | 0.10% Sodium Lauryl Sulfate | |
| | 15.00% Provesta 356 | |
| | 5.00% Provesta 400 | |
| | 2.00% PEG-3350 | |
| | 1.50% Magnesium Stearate | |
| | 0.004% Tenox 4 | |
| | 22.00% Glycerin | |
| | 8.50% Soybean Oil | |
| | 20.566% Dicalcium Phosphate | |
| PAH-19- | 8.33% Pimobendan & DiCal Coated Granules of | Pimo 5 mg SCT |
| 01-002 | any of Tables I-VI | SCT size = 1.20 g |
| | 20% Starch, fully pre-gelatinized | |
| | 5% Croscarmellose Sodium | |
| | 4% Povidone-K30 | |
| | 15% Provesta 356 | |
| | 1.5% Magnesium Stearate | |
| | 0.10% Sodium Lauryl Sulfate | |
| | 22% Glycerin | |
| | 9% Soybean Oil w/0.02% TBHQ | |
| | 2% PEG 3350 | |
| | 13.07% DiCal | |
| PAH-19- | 8.33% Pimobendan & Lactose Coated Granules | Pimo 5 mg SCT |
| 01-003 | of any of Tables I-VI | SCT size = 1.20 g |
| | 20% Starch, fully pre-gelatinized | |
| | 5% Croscarmellose Sodium | |
| | 4% Povidone-K30 | |
| | 15% Provesta 356 | |
| | 1.5% Magnesium Stearate | |
| | 0.10% Sodium Lauryl Sulfate | |
| | 22% Glycerin | |
| | 9% Soybean Oil w/0.02% TBHQ | |
| | 2% PEG 3350 | |
| | 13.07% DiCal | |
| PAH-19- | 5% Pimobendan & Lactose Coated Granules of | Pimo *2.5* mg SCT |
| 01-004 | any of Tables I-VT | SCT size = 1200 mg |
| | 20% Starch, fully pre-gelatinized | |
| | 5% Croscarmellose Sodium 5% Povidone-K30 | |
| | 6% Liver Powder | |
| | 5% Provesta 3495% Provesta 208 | |
| | 1.2% Magnesium Stearate | |
| | 23.5% Glycerin | |
| | 9.1% Soybean Oil | |
| | 0.004% Tenox 4 | |
| | 2% PEG 3350 | |
| | 13.2% DiCal | |
| PAH-19- | 10.02% Pimobendan & DiCal Coated Granules of | Pimo 5 mg SCT |
| 01-005 | any of Tables I-VI | SCT size = 1.20 g |
| | 20.0% Starch, fully pre-gelatinized | (1200 mg × 10% × 0.0416 = 5 mg) |
| | 5.0% Croscarmellose Sodium | |
| | 4.0% Povidone-K30 | |
| | 5.0% Liver Powder | |
| | 10.0% Provesta 356 | |
| | 1.5% Magnesium Stearate | |
| | 0.1% Sodium Lauryl Sulfate | |
| | 22.0% Glycerin | |
| | 9.5% Soybean Oil | |
| | 2.0% PEG 3350 | |
| | 10.88% DiCal | |
| PAH-19- | 10.02% Pimobendan & DiCal Coated Granules | Pimo 5 mg SCT |
| 01-006 | (8.33% theoretical) of any of Tables I-VI | SCT size = 1.20 g |
| | 20.0% Starch, fully pre-gelatinized | |
| | 5.0% Croscarmellose Sodium | |
| | 4.0% Povidone-K30 | |
| | 12.0% Provesta 356 | |
| | 1.5% Magnesium Stearate | |
| | 0.1% Sodium Lauryl Sulfate | |
| | 3.0% Liver Powder | |
| | 22.1% Glycerin | |
| | 10.0% Soybean Oil | |
| | 2.0% PEG 3350 | |
| | 10.28% DiCal | |
| PAH-19- | 4.49% Pimobendan Lactose Granules (8.985 | Pimo 5 mg in 2.4 g SCT |
| 01-007 | theoretical) of any of Tables I-VI | |
| | 20.0% Starch Lycotab Roquette | |
| | 5.0% Croscarmellose sodium | |
| | 4.0% Povidone K-30 | |
| | 12.0% Provesta 356 | |
| | 3.0% Liver Powder | |
| | 1.5% Magnesium Stearate | |
| | 22.3% Glycerin | |
| | 9.8% Soybean Oil w/TBHQ | |
| | 2.0% PEG3350 | |
| | 0.1% SLS Powder | |
| | 15.81% DiCal Powder | |
| PAH-19- | 5% Pimobendan Dical Granules of any of Tables | Pimo 5 mg in 2.4 g SCT |
| 01-008 | I-VI | (2400 mg × 5% × 0.0416 = 5 mg) |
| | 20.0% Starch Lycotab Roquette | |
| | 3.0% Croscarmellose sodium | |
| | 6.0% Povidone K-30 | |
| | 12.0% Provesta 356 | |
| | 3.0% Liver Powder | |
| | 1.5% Magnesium Stearate | |
| | 22.3% Glycerin | |
| | 9.8% Soybean Oil w/TBHQ | |
| | 2.0% PEG3350 | |
| | 0.05% SLS Powder | |
| | 15.35% DiCal Powder | |
| PAH-19- | 10.02% Pimobendan DiCal Granules (8.33 | Pimo 5 mg in 1200 mg |
| 01-009 | theoretical, 10.02 actual after correction for | SCT(1200 mg × 10% × 0.0416 = 5 mg) |
| | potency) of any of Tables I-VI | LIVER CHEESEBURGER |
| | 20.0% Starch Lycotab Roquette | |
| | 3.0% Croscarmellose sodium | |
| | 6 0% Povidone K-302.0% Provesta 208 (cheese) | |
| | 7.0% Provesta 400 | |
| | 3.0% Liver Powder | |
| | 1.5% Magnesium Stearate | |
| | 22.3% Glycerin | |
| | 9.8% Soybean Oil w/TBHQ | |
| | 2.0% PEG3350 | |
| | 13.38% DiCal Powder | |
| PAH-19- | Pimobendan or Granules of any of Tables 1-VI | LIVER CHEESEBURGER |
| 01-010 | 23.0% Starch Lycotab Roquette | (VEHICLE) |
| | 3.0% Croscarmellose sodium | |
| | 6.0% Povidone K-30 | |
| | 2.0% Provesta 208 (cheese) | |
| | 7.0% Provesta 400 | |
| | 3.0% Liver Powder | |
| | 1.5% Magnesium Stearate | |
| | 25% Glycerin | |
| | 9.8% Soybean Oil w/TBHQ | |
| | 2.0% PEG3350 | |
| | 17.7% DiCal Powder | |
| PAH-19- | 10.02% Pimobendan Dical Granules (8.33 | LIVER CHEESEBURGER |
| 01-011 | theoretical, 10.02 actual after correction for | |
| | potency) of any of Tables 1-VI | |
| | 23.0% Starch Lycotab Roquette | |
| | 3.0% Croscarmellose sodium | |
| | 6.0% Povidone K-30 | |
| | 2.0% Provesta 208 (cheese) | |
| | 7.0% Provesta 400 | |
| | 3.0% Liver Powder | |
| | 1.5% Magnesium Stearate | |
| | 25% Glycerin | |
| | 9.8% Soybean Oil w/TBHQ | |
| | 2.0% PEG3350 | |
| | 7.68% DiCal Powder | |
| PAH-19- | Pimobendan or Pimobendan Granules of any of | LIVER CHEESEBURGER |
| 01-012 | Tables 1-VI | (VEHICLE) |
| | 21.0% Starch Lycotab Roquette | |
| | 3.0% Croscarmellose sodium | |
| | 5.0% Povidone K-30 | |
| | 2.0% Provesta 208 (cheese) | |
| | 7.0% Provesta 400 | |
| | 3.0% Liver Powder | |
| | 1.5% Magnesium Stearate | |
| | 25% Glycerin | |
| | 9.8% Soybean Oil | |
| | 2.0% PEG3350 | |
| | 20.7% DiCal Powder | |

An exemplary formulation is as set forth in Table X below.

TABLE X
Formulation

| | |
|---|---|
| Pimobendan or Pimobendan Granules | LIVER |
| of any of Tables I-VT | CHEESEBURGER |
| 18-23% Starch | (VEHICLE) |
| 2-4% Croscarmellose sodium | |
| 4-6% Povidone K-30 | |
| 1-3% Provesta 208 (cheese) | |
| 6-8% Provesta 400 | |
| 2-4% Liver Powder | |
| 1-2% Magnesium Stearate | |
| 20-30% Glycerin | |
| 7-12% Soybean Oil | |
| 1-3% PEG3350 | |
| 18-23% DiCal Powder | |

An exemplary formulation is as set forth in Table XI below.

TABLE XI
Formulation

| Component | w/w% |
|---|---|
| Pimobendan coated granule of any of Tables I-VI | 5-15 |
| Starch (fully pregelatinized) | 15-25 |
| Crosscarmellose sodium | 1-7 |
| Polyvinylpyrrolidone | 2-7 |
| Provesta ® flavoring | 10-25 |
| Magnesium stearate | 0.5-3 |
| Glycerin | 15-30 |
| Soybean oil with 0.02% TBHQ | 5-15 |
| Polyethylene glycol 3350 | 0.5-3 |
| Dicalcium phosphate | 10-25 |
| | 100 |

The formulations of the disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a carrier suitable for administration via oral administration.

Also provided herein is a method of treating a cardiac disease or disorder in a subject by administering a therapeutically effective amount of a formulation of the disclosure.

In various embodiments cardiac disease or disorder is congestive heart failure, cardiomyopathy, dilated or restrictive cardiomyopathy or atrioventricular valvular insufficiency.

In the methods described herein, an appropriate dosage level of pimobendan will generally be about 0.01 to about 50 mg/kg, such as, for example, about 0.25 to about 15mg/kg per day, such as about 2.0 to about 14 mg/kg per day. Within this range the dosage of each active ingredient may be about 0.25 to 3.5 mg/kg, 0.25 to 14 mg/kg, 1.0 to 10 mg/kg, 1.5 to 10 mg/kg, 2.0 to 10 mg/kg, 2.5 to 8.0 mg/kg, 2.5 to 8 mg/kg, 2.5 to 7.0 mg/kg, 2.5 to 6.5 mg/kg, 2.5 to 6.0 mg/kg, 2.5 to 5.5 mg/kg, 2.5 to 5.0 mg/kg, 2.5 to 4.0 mg/kg, 2.5 to 3.5 mg/kg (including all intermediate dosages, such as 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, etc. mg/kg), in a single dosage form. The formulations of the disclosure are particularly useful in mammals, especially companion animals, and most especially cats and dogs.

In various embodiments, the oral formulation may be administered on a daily basis throughout the duration of the cardiac disease or disorder.

The following examples are provided to further illustrate the embodiments of the present invention, but are not intended to limit the scope of the invention. While they are typical of those that might be used, other procedures, methodologies, or techniques known to those skilled in the art may alternatively be used.

### Example I

### Formulation Manufacture and Stability

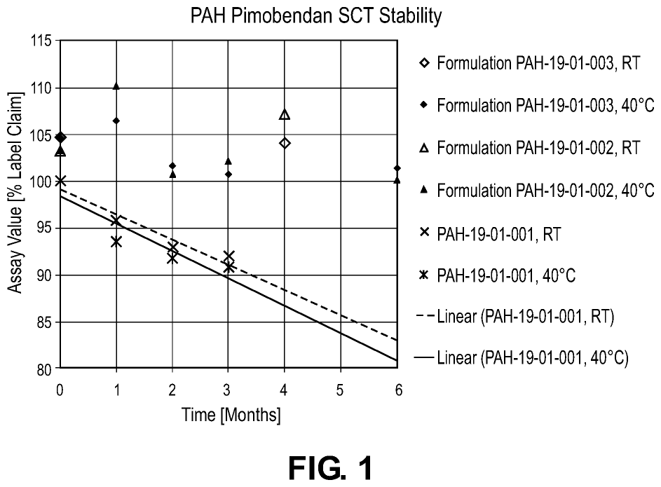

The formulations of Tables VII (PAH 19-01-002 of FIG. 1) and VIII (PAH 19-01-003 of FIG. 1) were prepared and tested for stability.

To make the granules, the binder polyvinylpyrrolidone is dissolved in a suitable solvent (most often used are ethanol or water). The Pimobendan API and lactose monohydrate or dicalcium phosphate are dry blended. While continuing to mix the dry blend, the binder solution is added slowly. Once the solution is added and the mixture does not have large clumps, discontinue mixing and pass the granules through a suitable mesh screen (a #20 mesh) which produces appropriately sized granules. At this point the granules are still wet. Dry the granules, or allow the granules to dry (e.g., overnight). Then move the granules to a fluid bed operation where the appropriate level (e.g., a 5% weight gain) of coating (Kollicoat® IR) is applied. Once the spraying is complete and the coated granules are dry, pass the coated granules through a suitable mesh screen (#20 mesh). Assay the granules for the content of the API.

To make the soft chewable tablets, heat the PEG 3350 to 65-75° C. to cause it to melt. Meanwhile, weigh the remaining dry ingredients and pass them through though a suitable screen to break up any clumps, include the right amount of granules, correcting for the assay of the granules. Mix the dry ingredients (e.g., in a V-blender). Transfer the dry blend to a suitable blender and while mixing add the glycerin at a suitable rate known to subject matter experts. While mixing, add the Soybean Oil with 0.02% TBHQ at a suitable rate. While mixing, add the liquefied PEG 3350 at a suitable rate. Allow the final blend to mix for a suitable amount of time. Mold the product into the final tablet form and allow the tablet to cure.

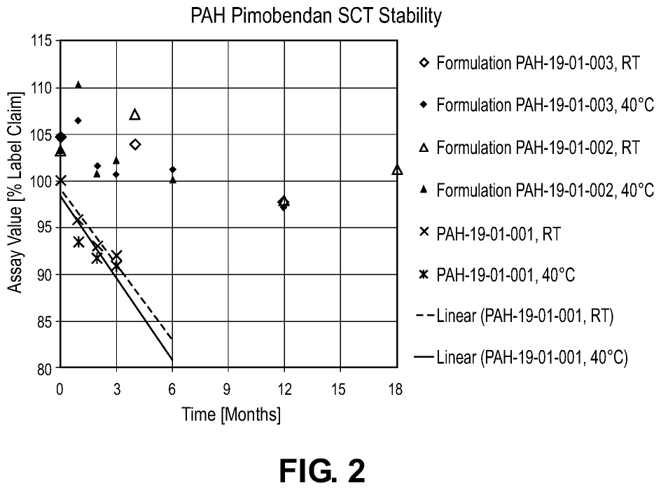

The formulations were then tested for stability. FIG. 1 shows stability of the formulations of Tables VII (PAH 19-01-002 of FIGS. 1-2) and VIII (PAH 19-01-003 of FIGS. 1-2) as compared to an equivalent formulation in which the granules are not coated (PAH 19-01-001 of FIGS. 1-2). The formulations of Tables VII and VIII are expected to remain stable for at least 6 months.

Although the objects of the disclosure have been described with reference to the above example, it will be understood that modifications and variations are encompassed within the spirit and scope of the disclosure. Accordingly, the disclosure is limited only by the following claims.

What is claimed is:

1. A single dose, chewable oral formulation comprising:
a) pimobendan or a pharmaceutically acceptable salt or solvate thereof; and
b) a pharmaceutically acceptable carrier,
wherein the pimobendan is in a granular form and coated with a polyvinyl alcohol-polyethylene glycol graft copolymer.

2. The formulation of claim 1, wherein 1.25 mg, 2.5 mg, 5 mg, 10 mg, 12.5 mg, or 15 mg of pimobendan is present in the formulation.

3. The formulation of claim 1, wherein granules of the granular particulate have a dimension of an average size between about 100 and 1500 μm.

4. The formulation of claim 1, wherein the formulation is stable for at least 6, 12, 24 or 36 months at 25° C. or 40° C.

5. The formulation of claim 1, wherein the pimobendan is granulated with lactose and/or dicalcium phosphate and then coated.

6. The formulation of claim 5, wherein the pharmaceutically acceptable carrier is a homogeneous mixture comprising:
i) starch;
ii) croscarmellose sodium;
iii) sodium lauryl sulfate;
iv) magnesium stearate;
v) dicalcium phosphate;
vi) polyethylene glycol;
vii) glycerin; and
viii) soybean oil.

7. The formulation of claim 5, wherein the pharmaceutically acceptable carrier is a homogeneous mixture comprising:
i) starch;
ii) croscarmellose sodium;
iii) polyvinylpyrrolidone;
iv) magnesium stearate;
v) dicalcium phosphate;
vi) polyethylene glycol;
vii) glycerin; and
viii) soybean oil.

8. The formulation of claim 7, wherein the starch is selected from the group consisting of native starch, gelatinized starch, partly gelatinized starch, starch powder, starch granules, chemically modified starch, swellable physically modified starch and mixtures thereof.

9. The formulation of claim 8, wherein the starch is gelatinized starch.

10. The formulation of claim 7, further comprising flavoring.

11. The formulation of claim 7, further comprising an antioxidant.

12. The formulation of claim 7, further comprising polyvinylpyrrolidone.

13. The formulation of claim 7, wherein the formulation is a tablet or capsule.

14. A method of treating a cardiac disorder in a subject comprising administering a therapeutically effective amount of a formulation claim 1 to a subject.

15. The method of claim 14, wherein the subject is a mammal.

16. The method of claim 15, wherein the subject is a canine.

17. The method of claim 15, wherein the subject is a feline.

18. The method of claim 14, wherein the cardiac disorder is congestive heart failure.

19. The method of claim 14, wherein the cardiac disorder is cardiomyopathy.

20. The method of claim 19, wherein the cardiac disorder is dilated or restrictive cardiomyopathy.

21. The method of claim 14, wherein the cardiac disorder is atrioventricular valvular insufficiency.
